(19) United States
(12) Patent Application Publication
(10) Pub. No.: US 2015/0087987 A1
(43) Pub. Date: Mar. 26, 2015

(54) MOBILE ULTRASOUND DIAGNOSIS PROBE APPARATUS FOR USING TWO-DIMENSIONAL ARRAY DATA, MOBILE ULTRASOUND DIAGNOSIS SYSTEM USING THE SAME

(71) Applicant: HEALCERION CO., LTD., Seoul (KR)

(72) Inventors: Jeong Won Ryu, Seoul (KR); You Chan Choung, Seoul (KR)

(21) Appl. No.: 14/394,229

(22) PCT Filed: Apr. 23, 2013
(86) PCT No.: PCT/KR2013/003439

§ 371 Date: Oct. 13, 2014

(30) Foreign Application Priority Data
Apr. 23, 2012 (KR) 10-2012-0041904

(51) Int. Cl. A61B 8/08; A61B 8/00; A61B 8/14

(52) U.S. Cl. CPC A61B 8/5207; A61B 8/14; A61B 8/4427; A61B 8/4488; A61B 8/5269; A61B 8/56
USPC 600/447

(57) ABSTRACT

Provided is a mobile ultrasound diagnosis probe apparatus including a transmission signal forming unit forming a transmission signal for obtaining a frame of an ultrasound image, an ultrasound probe transducing the transmission signal of the transmission signal forming unit into an ultrasound signal, transmitting the ultrasound signal to an object, and obtaining analog ultrasound data reflected from the object, a two-dimensional array processor adjacently arranging the obtained analog ultrasound data compensated with respect to time gains thereof and adjusted with respect to intensity and contrast thereof for each ultrasound vector to be processed into two-dimensional array ultrasound data, a compressor compressing the two-dimensional array ultrasound data adjacently arranged for each ultrasound vector, and a wireless communication unit wirelessly transmitting the compressed two-dimensional array ultrasound data to an ultrasound diagnostic apparatus.

### TECHNICAL FIELD

The present invention relates to a mobile ultrasound diagnosis probe apparatus and a mobile ultrasound diagnosis system using the same, and more particularly, to a mobile ultrasound diagnosis probe apparatus processing ultrasound data obtained from an object into two-dimensional array data and compressing and wirelessly transmitting the two-dimensional array data and a mobile ultrasound diagnosis system using the apparatus.

### BACKGROUND ART

Since having noninvasive and nondestructive properties, ultrasound diagnosis systems are generally used in the medical field to obtain information of the inside of an object. Since surgical operations of directly incising and observing objects are un-necessary and high-resolution images of internal organizations of objects may be provided to doctors, ultrasound diagnosis systems are very importantly used in the medical field.

Generally, an ultrasound system includes an ultrasound probe, a beam former, a data processor, a scan transducer, and a display unit. The ultrasound probe transmits an ultrasound signal to an object and forms a reception signal by receiving a reflected ultrasound signal, that is, an ultrasound echo signal. The ultrasound probe includes at least one transducer element operating to transduce an ultrasound signal and an electric signal into one another. The beam former analog/digital-transduces the reception signal provided from the ultrasound probe, delays a time of a digital signal considering a position and a focusing point of each transducer element, and forms ultrasound data, that is, radio frequency (RF) data by summing the time-delayed digital signals up. The data processor performs various data processes with respect to ultrasound data, which are necessary for forming an ultrasound image. The scan transducer scan-transduces the processed ultrasound data to be displayed on a display area of the display unit. The display unit displays the scan-transduced ultrasound data as an ultrasound image on a screen.

Typically, data processing such as a time gain compensation (TGC) process, a plurality of finite impulse response (FIR) filtering processes, a plurality of decimation processes, an in-phase/quadrature-phase (I/Q) data forming process, and a compression process and a scan transducing process are sequentially performed. Due thereto, not only a lot of time is consumed to process a large amount of ultrasound data but also a frame rate is deteriorated.

### DISCLOSURE OF INVENTION

### Technical Problem

The present invention provides a mobile ultrasound diagnosis probe apparatus processing ultrasound data obtained from an object into two-dimensional array data and compressing and wirelessly transmitting the two-dimensional array data and a mobile ultrasound diagnosis system using the apparatus.

### Solution to Problem

To achieve these and other advantages and in accordance with the purpose of the present invention, as embodied and broadly described, there is provided a

According to another aspect of the present invention, there is provided mobile ultrasound diagnosis probe apparatus including a transmission signal forming unit forming a transmission signal for obtaining a frame of an ultrasound image, an ultrasound probe transducing the transmission signal of the transmission signal forming unit into an ultrasound signal, transmitting the ultrasound signal to an object, and obtaining analog ultrasound data reflected from the object, a two-dimensional array processor adjacently arranging the obtained analog ultrasound data compensated with respect to time gains thereof and adjusted with respect to intensity and contrast thereof for each ultrasound vector to be processed into two-dimensional array ultrasound data, a compressor compressing the two-dimensional array ultrasound data adjacently arranged for each ultrasound vector, and a wireless communication unit wirelessly transmitting the compressed two-dimensional array ultrasound data to an ultrasound diagnostic apparatus.

The two-dimensional array processor may arrange received ultrasound vectors in a serial stream to be adjacent for each ultrasound vector unit lengthwise to be processed into the two-dimensional array ultrasound data.

The mobile ultrasound diagnosis probe apparatus may further include a beam former generating digitalized ultrasound data by using the analog ultrasound data obtained from the ultrasound probe, a time gain compensation (TGC) unit compensating the digitalized ultrasound data with time gains, and an intensity and contrast adjuster adjusting intensity and contrast of the ultrasound image.

The mobile ultrasound diagnosis probe apparatus may further include a TGC unit compensating the analog ultrasound data obtained from the ultrasound probe with time gains, a beam former generating digitalized ultrasound data by using the ultrasound data compensated with time gains, and an intensity and contrast adjuster adjusting intensity and contrast of the ultrasound image.

The beam former may include M number of arrays whose size is N when using M number of ultrasounds for one ultrasound image frame and performing N times of sampling each ultrasound that is reflected from the object and returns. The TGC unit may compensate the ultrasound data according to a TGC table.

The intensity and contrast adjuster may adjust an intensity value of a certain value or less to 0 and may adjust an intensity value of a certain value or more to a maximum value. The intensity and contrast adjuster may adjust a contrast value of a certain value or less to 0 and may adjust a contrast value of a certain value or more to a maximum value.

The two-dimensional array processor may generate two-dimensional ultrasound data including N×M arrays when using M number of ultrasounds for one ultrasound image frame and performing N times of sampling each ultrasound that is reflected from the object and returns.

The wireless communication unit may include short-distance wireless communication by using one of Bluetooth, a wireless universal serial bus (USB), a wireless local area network (LAN), wireless fidelity (WiFi), Zigbee, and infrared data association (IrDA).

According to another aspect of the present invention, there is provided a mobile ultrasound diagnostic system including a mobile ultrasound diagnosis probe apparatus, which is portable, digital-processes ultrasound data obtained from an object, compensates the digitalized ultrasound data with time gains, adjusts intensity and contrast of the ultrasound data, processes the ultrasound data into two-dimensional array ultrasound data by adjacently arranging for each ultrasound vector, compresses the two-dimensional array ultrasound data, and wirelessly transmits the two-dimensional array ultrasound data, and an ultrasound diagnostic apparatus, which receives the two-dimensional array ultrasound data from the mobile ultrasound diagnosis probe apparatus and generates ultrasound image data for diagnosis by releasing compression of the two-dimensional array ultrasound data.

The mobile ultrasound diagnosis probe apparatus may arrange received ultrasound vectors in a serial stream to be adjacent for each ultrasound vector unit lengthwise to be processed into the two-dimensional array ultrasound data.

The ultrasound diagnostic apparatus may determine an ultrasound measurement depth according to an input of a user and may transmit a parameter for adjusting time gains and a parameter for adjusting the intensity and the contrast to the mobile ultrasound diagnosis probe apparatus.

The ultrasound diagnostic apparatus may transmit dummy data for automatically measuring a wireless communication environment and for determining the size of transmission data to the mobile ultrasound diagnosis probe apparatus, and the mobile ultrasound diagnosis probe apparatus may receive the dummy data from the ultrasound diagnostic apparatus, may calculate an available band of wireless communication currently used by measuring an amount of time used to receive the dummy data, and may determine the size of data to be wirelessly transmitted, according to the available band.

### ADVANTAGEOUS EFFECTS OF INVENTION

According to the present invention, According to the present invention, since a throughput of processing ultrasound data may be more reduced than processing image data by time gain compensation, intensity and contrast adjustment, and two-dimensional array data processing operation in a mobile ultrasound diagnosis probe apparatus, programs operated in an ultrasound diagnostic apparatus may be simplified and consumption of resources such as a memory and a central processing unit (CPU). In addition, the ultrasound diagnostic apparatus may be applied to a mobile device having relatively low specifications.

### BRIEF DESCRIPTION OF DRAWINGS

FIG. 1 is a block view illustrating a mobile ultrasound diagnosis system according to an embodiment of the present invention;

FIG. 2 is a view illustrating a transmitting ultrasound vector of an ultrasound probe according to an embodiment of the present invention;

FIG. 3 is a view illustrating ultrasound data when M number of ultrasounds are used and N times of samplings are performed;

FIG. 4 is a view illustrating a time gain compensation operation according to an embodiment of the present invention;

FIG. 5 is a view illustrating intensity adjustment according to an embodiment of the present invention;

FIG. 6 is a view illustrating contrast adjustment according to an embodiment of the present invention;

FIG. 7 is a view illustrating two-dimensional arrangement according to an embodiment of the present invention;

FIGS. 8 to 10 are views illustrating a two-dimensional arrangement process according to an embodiment of the present invention; and

FIGS. 11 to 14 are views illustrating a two-dimensional arrangement process according to another embodiment of the present invention.

### BEST MODE FOR CARRYING OUT THE INVENTION

Hereinafter, the embodiments of the present invention will be described in detail with reference to the attached drawings. The following embodiments are provided as examples to fully convey the spirits of the present invention to a person skilled in the art. Accordingly, the present invention is not limited to the embodiments described below and may be embodied in other forms. Also, in the drawings, a width, a length, and a thickness of an element may be exaggerated for convenience of description. Like reference numerals designate like elements throughout.

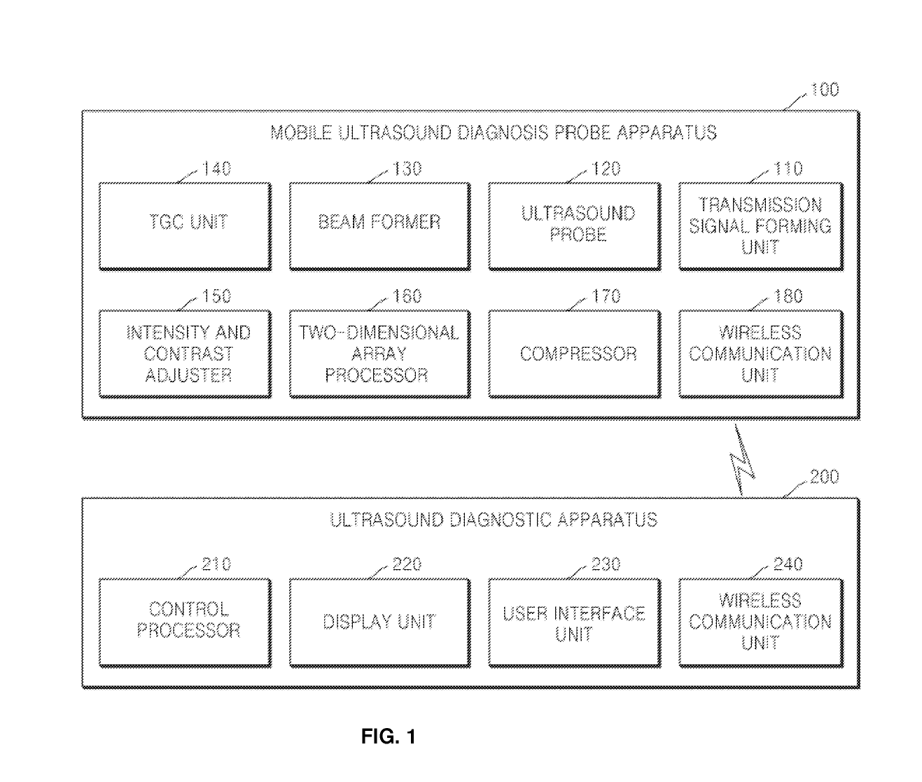

FIG. 1 is a block view illustrating a mobile ultrasound diagnosis system according to an embodiment of the present invention.

Referring to FIG. 1, the ultrasound diagnosis system may include a mobile ultrasound diagnosis probe apparatus 100 and an ultrasound diagnostic apparatus 200.

The mobile ultrasound diagnosis apparatus 100 may include a transmission signal forming unit 110, an ultrasound probe 120 including a plurality of transducer elements, a beam former 130, a time gain compensation (TGC) unit 140, an intensity and contrast adjuster 150, a two-dimensional array processor 160, a compressor 170, and a wireless communication unit 180.

The transmission signal forming unit 110 forms a plurality of transmission signal for obtaining a frame of an ultrasound image by considering the transducer elements of the ultrasound probe 120 and a focusing point. The frame is formed of a plurality of scan lines. Also, the ultrasound image may include a brightness mode (B-mode) image in which a reflection coefficient of an ultrasound echo signal reflected from an object is shown as a two-dimensional image, a Doppler mode (D-mode) image in which the velocity of a moving object is shown as a Doppler spectrum by using a Doppler effect, a color mode (C-mode) image in which the velocity of a moving object and a scatterer are shown in color by using the Doppler effect, an elastic mode (E-mode) image in which a difference between mechanical responses of a medium when applying stress to an object or not is shown as an image, and a three-dimensional mode (3D-mode) image in which a reflection coefficient of an ultrasound echo signal reflected from an object is shown as a 3D image.

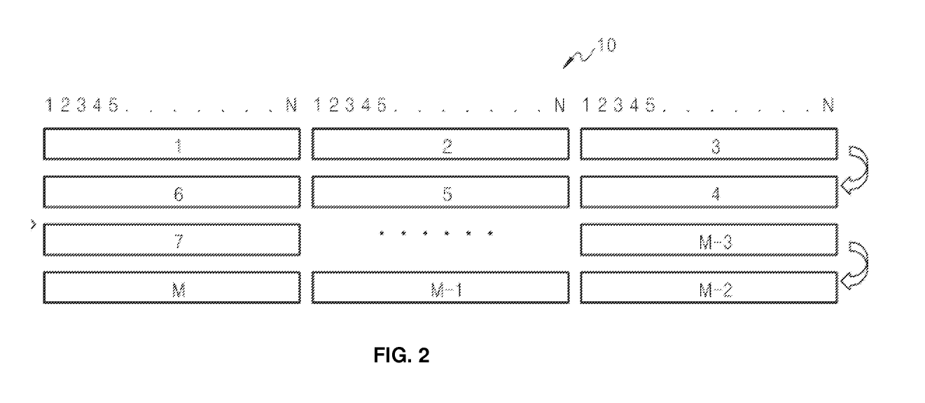

The ultrasound probe 120, as shown in FIG. 2, transduces the transmission signal provided from the transmission signal forming unit 110 into an ultrasound signal and transmit the ultrasound signal to an object. The ultrasound probe 120 receives the ultrasound echo signal reflected from the object and forms a reception signal. The ultrasound probe 120 forms a plurality of reception signals by repetitively performing transmission and reception of the ultrasound signals by using the plurality of transmission signals. In this case, since having vector data, the ultrasound signals transmitted and received by the ultrasound probe 120 are designated as ultrasound vectors. For example, ultrasound vectors transmitted from the ultrasound probe 120 to a human body are designated as transmission ultrasound vectors, and ultrasound vectors echoed from the human body to the ultrasound probe 120 are designated as reception ultrasound vectors.

In the present embodiment, the ultrasound probe 120 may be embodied as a convex probe, a linear probe, a 3D dimensional probe, a trapezoidal probe, and an intravascular ultrasound (IVUS) probe.

The beam former 130 analog/digital-transduces the plurality of reception signals provided from the ultrasound probe 120 and generates digitalized ultrasound data. In addition, the beam former 130, considering the transducer elements of the ultrasound probe 120 and the focusing point, receives and focuses a plurality of digital-transduced reception signals and forms a plurality of digital reception focusing beams. In the present embodiment, the beam former 130, to improve the speed of processing reception signals, may be embodied as a field programmable gate array (FPGA) or an application specific integrated circuit (ASIC).

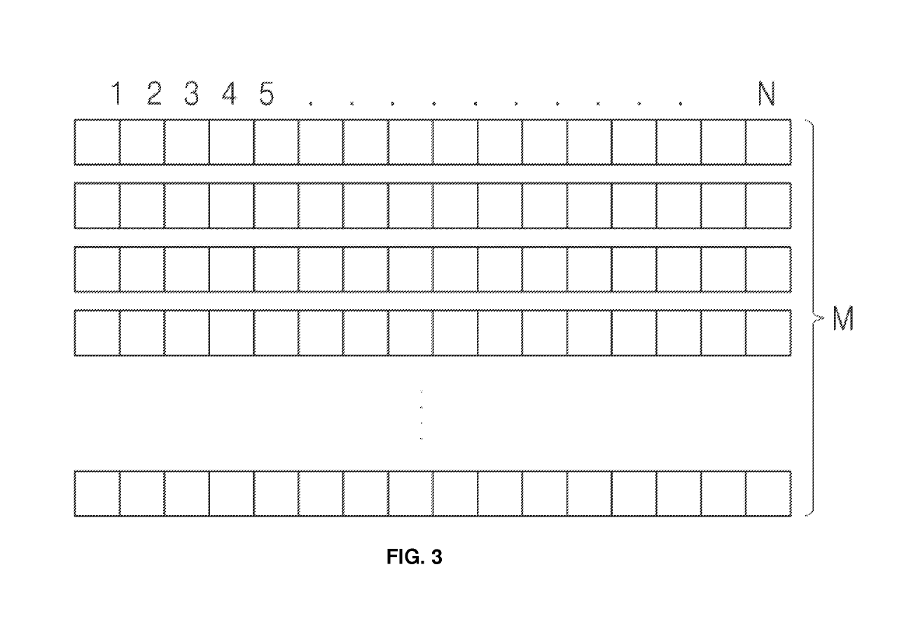

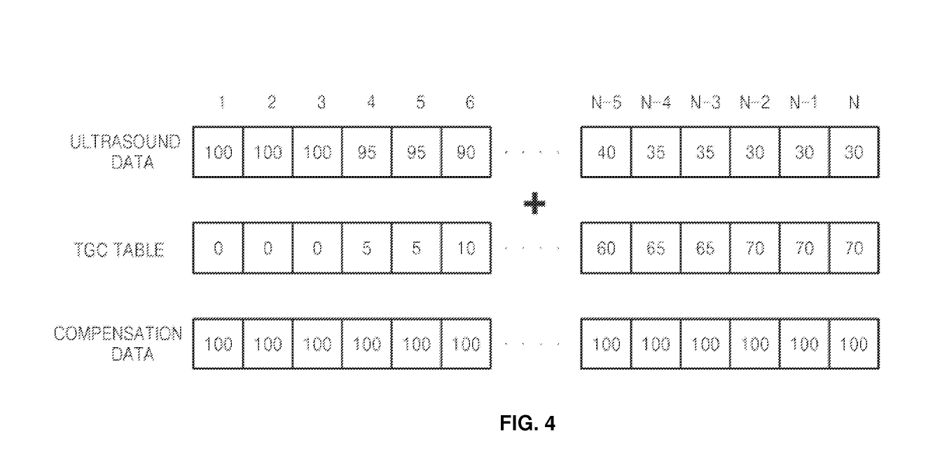

The digitalized ultrasound data, as shown in FIG. 3, are data stored as an array capable of being shown as intensity values in the ultrasound image. The size of the array is determined according to the number of sampling ultrasounds that are reflected by the human body and return. The number of the arrays for each ultrasound image may be determined according to the number of ultrasound used to form each ultrasound image. When M number of ultrasounds are used for each ultrasound image and N times of sampling each ultrasound that is reflected by the human body and returns are performed, M number of arrays whose size if N may be generated.

The TGC unit 140, as shown in FIG. 3, compensates time gains of the digitalized ultrasound data.

Since ultrasound is absorbed in the human body due to properties thereof, ultrasound reflected by a deeper position and returns later is reduced in size because a greater loss of energy occurs. In the same human body, the size of ultrasound data reflected by the deeper position is relatively smaller. Accordingly, it is necessary to compensate with a greater value in proportion to a time for being reflected and returning. When using the ultrasound data array whose size is N, a compensation value is determined by generating a time gain compensation table having the same size and is added to an ultrasound data array value.

The intensity and contrast adjuster 150 adjusts the intensity and the contrast of the ultrasound image. When the intensity and contrast adjuster 150 lowers an intensity value, an intensity value of a certain value or less is changed into 0. When the intensity and contrast adjuster 150 makes an intensity value higher, an intensity value or a certain value or more is changed into a maximum value.

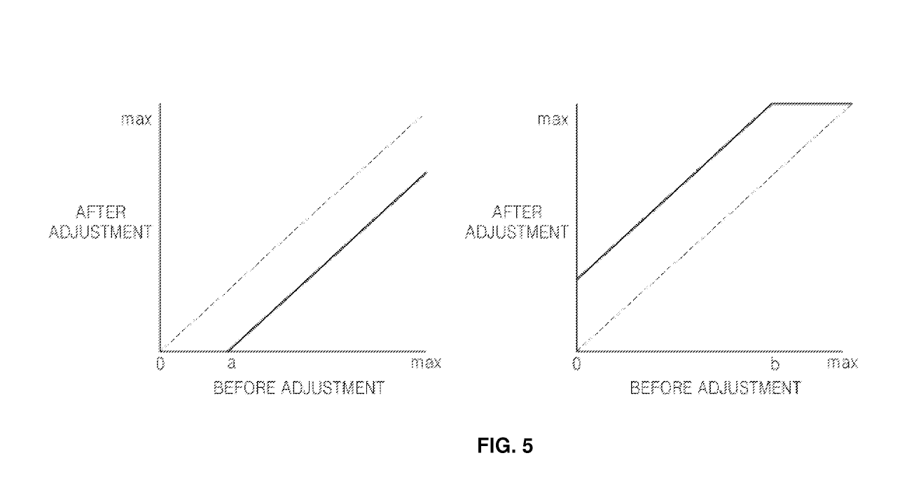

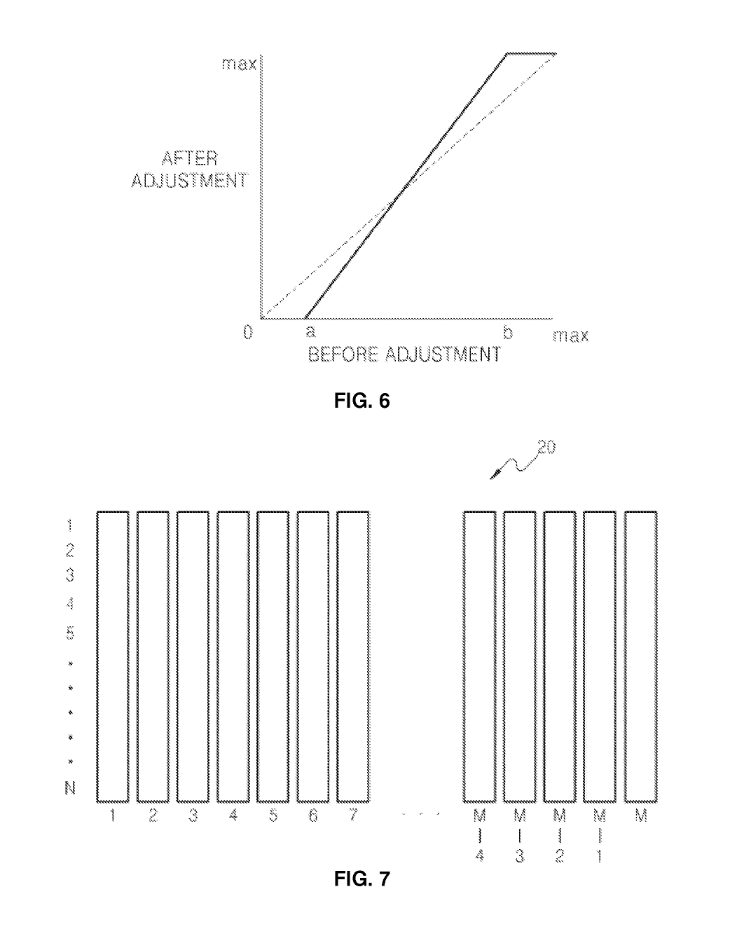

Accordingly, referring to FIGS. 5 and 6, an intensity value smaller than a is adjusted into 0 when the intensity value is lowered by intensity value adjustment of the intensity and contrast adjuster 150 and an intensity value greater than b is adjusted into the maximum value.

The intensity and contrast adjuster 150 may adjust the contrast of the ultrasound image. When the intensity and contrast adjuster 150 adjusts the contrast, the contrast in an intensity area having the importance in the ultrasound image is emphasized and other areas may be made as 0 or the maximum value.

Accordingly, when the intensity and contrast adjuster 150 adjusts the contrast, as shown in FIG. 5, a contrast difference increases while the intensity value exists within a range from a to b, is changed into 0 while being smaller than a, and is changed into the maximum value while being greater than b.

According to the operations of the TGC unit 140 and the intensity and contrast adjuster 150, the ultrasound data may be changed into 0 or the maximum value. Accordingly, when the same values are more generated, efficiency of a subsequent compression process becomes higher.

As described above, the ultrasound data are processed and wirelessly transmitted by the TGC unit 140 and the intensity and contrast adjuster 150, thereby simplifying a program operated in the ultrasound diagnostic apparatus 200 and reducing consumption of resources such as a memory and a CPU. The ultrasound diagnostic apparatus 200 may be applied to a mobile device whose specifications are relatively low.

The two-dimensional array processor 160 processes the ultrasound data whose time gains are compensated and intensity and contrast are adjusted into two-dimensional array ultrasound data. The two-dimensional array processor 160 may form a two-dimensional array by adjacently arranging received ultrasound vectors echoed from the human body.

The two-dimensional array processor 160 may arrange the reception ultrasound vectors echoed from the human body, for example, adjacently lengthwise, instead of forming an image by collecting the reception ultrasound vectors. The two-dimensional array processor 160 provides the compressor 170 the respective reception ultrasound vectors adjacently arranged, to be compressed.

The reception ultrasound vectors echoed from the human body are processed by the two-dimensional array processor 160 to be adjacently arranged instead of being collected to form an image in such a way that continuity of image patterns is increased and additionally the size of data becomes smaller relatively to image data. When the size of data to be processed becomes smaller, data to be processed in a compression process performed by the compressor 170 may be reduced as that.

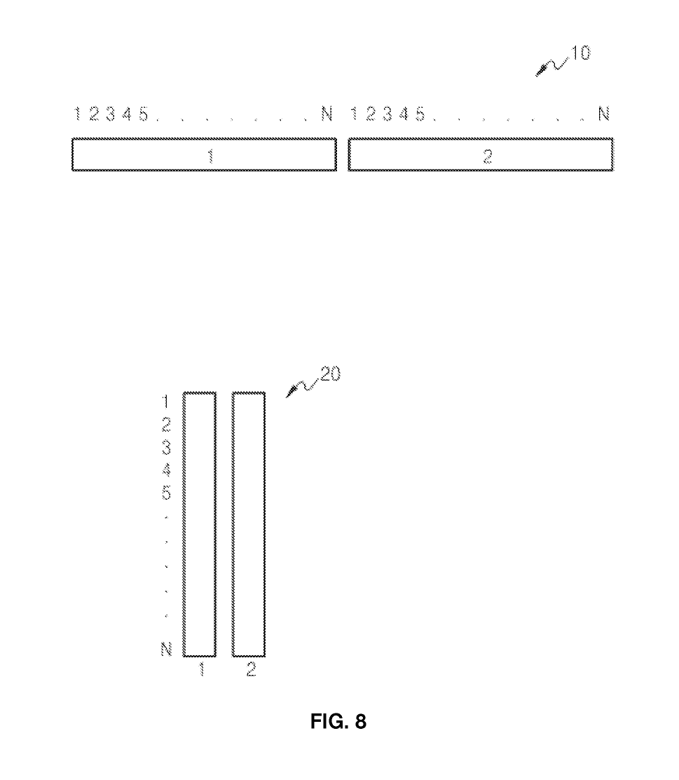

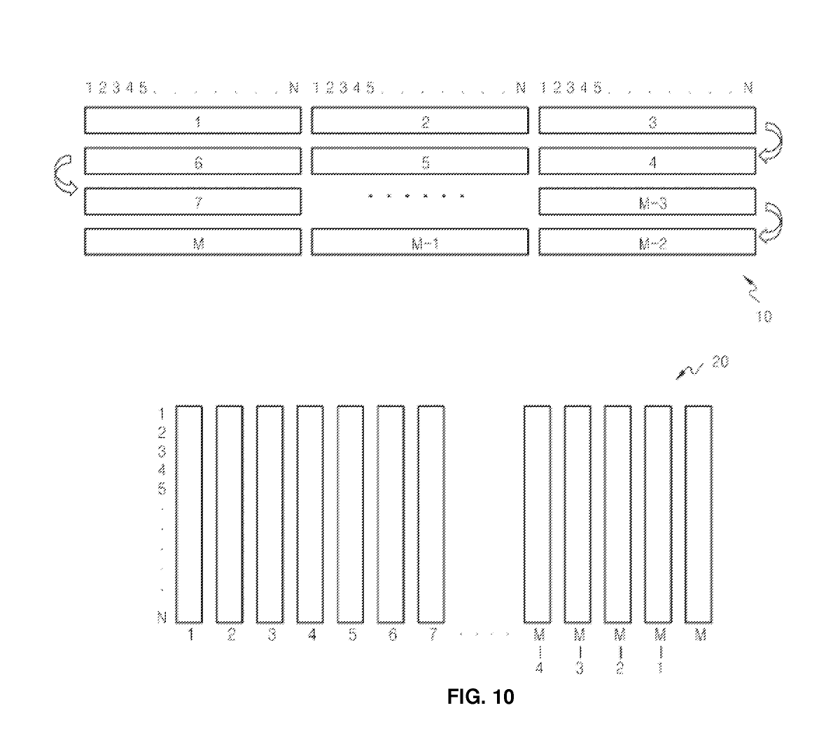

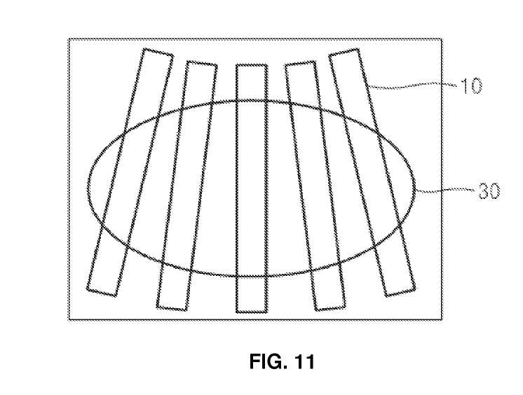

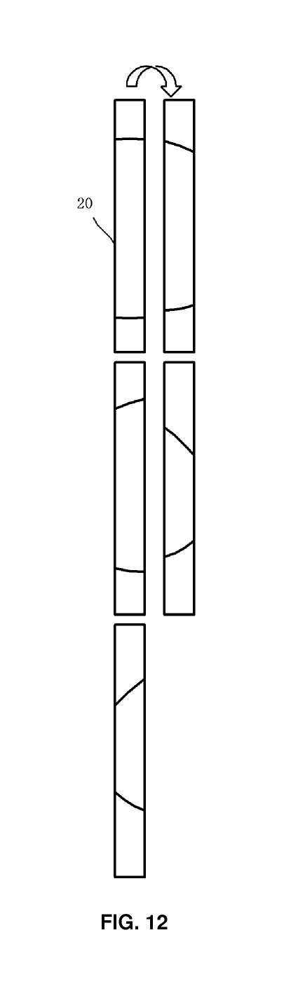

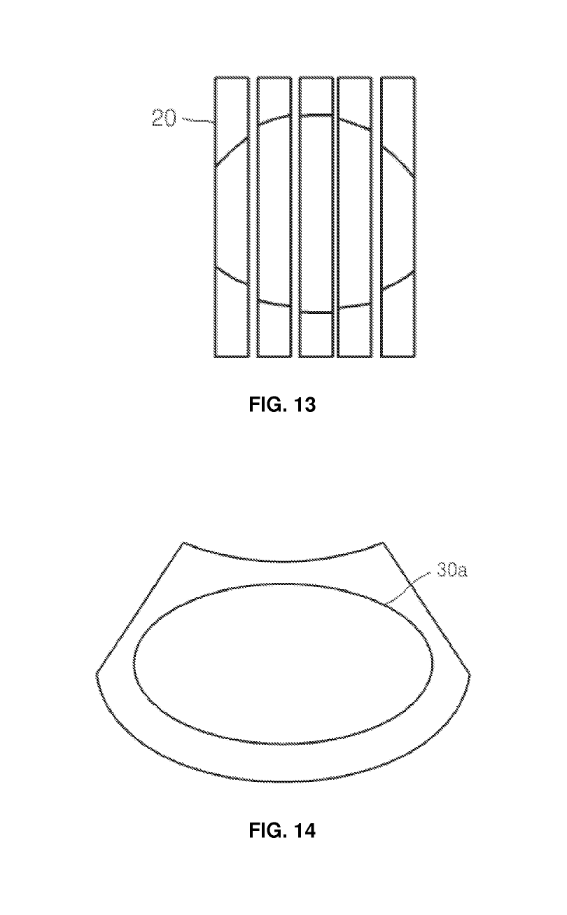

FIGS. 8 and 10 are views illustrating a two-dimensional arrangement process according to an embodiment of the present invention.

Referring to FIG. 8, the ultrasound probe 120 sequentially sends a first transmission ultrasound vector and a second transmission ultrasound vector to a human body. A reference numeral 10 indicates transmission ultrasound vectors. In addition, the ultrasound probe 120 receives a first reception ultrasound vector and a second reception ultrasound vector echoed from the human body. A reference numeral 20 indicates reception ultrasound vectors. The two-dimensional array processor 160 arranges the echoed first reception ultrasound vector and second reception ultrasound vector to be adjacent to each other lengthwise.

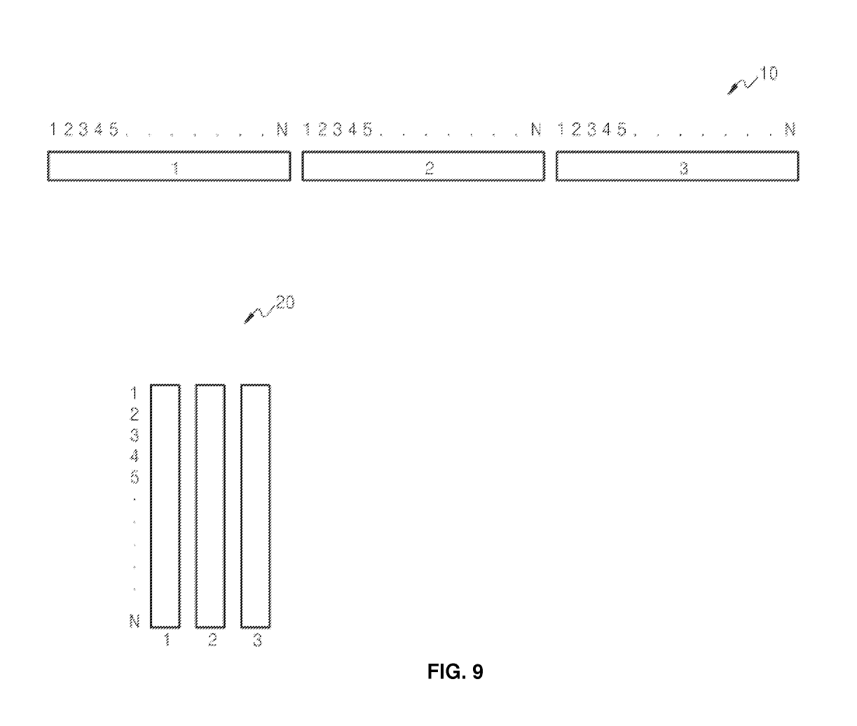

Referring to FIG. 9, the ultrasound probe 120 sends a third transmission ultrasound vector to the human body. In addition, the ultrasound probe 120 receives a third reception ultrasound vector echoed from the human body. The two-dimensional array processor 160 arranges the third reception ultrasound vector to be adjacent to the second reception ultrasound vector lengthwise.

Referring to FIG. 10, the ultrasound probe 120 sequentially emits an Mth transmission ultrasound vector to the human body. In addition, the ultrasound probe 120 receives an Mth reception ultrasound vector echoed from the human body. The two-dimensional array processor 160 arranges the Mth reception ultrasound vector to be adjacent to an M-1th reception ultrasound vector lengthwise.

In a modified example, the beam former 130 may include a two-dimensional array processing function and may generate an array for storing initial ultrasound data as a two-dimensional array.

A reason of applying the two-dimensional array is to compress data with a high compression rate. When ultrasound are compressed into a stream type in which one dimensional arrays are sequentially disposed, since compression is performed by using before and after values in order, a compression rate thereof is not high. For example, the compression rate may have a size of 60% of an original as an average. However, when image compression technology is used by two-dimensionally arranging using the two-dimensional array processor 160, since it is possible to use all peripheral values, compression into a size of 30% of the original may be performed in the case of lossless compression. When applying loss compression such as JPEG method, a different becomes greater.

The compressor 170 compresses ultrasound data to be transmitted to the ultrasound diagnostic apparatus 200. To effectively using a limited band under a wireless communication environment, compression is needed. The compressor 170 compresses the two-dimensional array data generated by the two-dimensional array processor 160. Accordingly, the compressor 170 may increase a compression rate by using the image compression technology instead of data compression. The compressor 170 may use both lossless compression and loss compression according to purpose of use and a wireless communication method.

The wireless communication unit 180 wirelessly transmits data compressed by the compressor 170 to the ultrasound diagnostic apparatus 200.

The wireless communication unit 180 may include, for example, short-distance wireless communication by using one of Bluetooth, a wireless universal serial bus (USB), a wireless local area network (LAN), wireless fidelity (WiFi), Zigbee, and infrared data association (IrDA).

The ultrasound diagnostic apparatus 200 has a wireless communication function and a display unit and may include various devices capable of operating application programs. For example, there may be a personal computer, a smart phone, a tablet type device, a pad type device, and personal digital assistants.

The ultrasound diagnostic apparatus 200 may include a control processor 210, a display unit 220, a user interface unit 230, and a wireless communication unit 240.

The control processor 210 receives ultrasound data from the mobile ultrasound diagnosis probe apparatus 100 via the wireless communication unit 240. The control processor 210 releases compression of the received ultrasound data by using the same method as that used by the mobile ultrasound diagnosis probe apparatus 100 and obtains two-dimensional array data. The control processor 210 generates an ultrasound image available to be displayed on a screen of the display unit 220 by using the released two-dimensional array data. The control processor 210 determines the size of the ultrasound image by considering the size of the screen of the display unit 220.

The control processor 210 may receive an input of a user via the user interface unit 230 and may forward the input to the mobile ultrasound diagnosis probe apparatus 100 by using wireless communication.

The control processor 210 determines, according to the input of the user, an ultrasound measurement depth, a parameter used by the TGC unit 140, and a degree of adjustment of the intensity and contrast adjuster 150.

The control processor 210 may determine whether to automatically measure a wireless communication environment and may determine the size of transmission data. The control processor 210 transmits dummy data having a certain size to the mobile ultrasound diagnosis probe apparatus 100.

Accordingly, the wireless communication unit 180 of the mobile ultrasound diagnosis probe apparatus 100 receives the dummy data from the ultrasound diagnostic apparatus 200 and calculates an available band of wireless communication currently used by measuring an amount of time used for receiving the dummy data.

The wireless communication unit 180 of the mobile ultrasound diagnosis probe apparatus 100 determines the size of data to be wirelessly transmitted, according to the available band. When the band is smaller, a rate of frames to be transmitted becomes reduced.

While the present invention has been particularly shown and described with reference to exemplary embodiments thereof, it will be understood by those of ordinary skill in the art that various changes in form and details may be made therein without departing from the spirit and scope of the present invention as defined by the following claims.

For example, in the present embodiment, it has been described that a transmission signal of a transmission signal forming unit in an ultrasound probe is transduced into an ultrasound signal and transmitted to an object, analog ultrasound data reflected from the object are obtained, and digitalized ultrasound data is generated by a beam former using the obtained analog ultrasound data and compensated with respect to time gains by a TGC unit. However, the present invention is not limited thereto. In another modified example, a transmission signal of a transmission signal forming unit in an ultrasound probe is transduced into an ultrasound signal and transmitted to an object, analog ultrasound data reflected from the object are obtained, the obtained analog ultrasound data are compensated with respect to time gains by a TGC unit, and digitalized ultrasound data may be generated by a beam former using the analog ultrasound data compensated with respect to the time gains.

### SEQUENCE LISTING FREE TEXT

100: a mobile ultrasound diagnosis probe apparatus

110: a transmission signal forming unit

120: an ultrasound probe

130: a beam former

140: a time gain compensation (TGC) unit

150: an intensity and contrast adjuster

160: a two-dimensional array processor

170: a compressor

180: a wireless communication unit

200: an ultrasound diagnostic apparatus

210: a control processor

220: a display unit

230: a user interface unit

240: a wireless communication unit

1. A mobile ultrasound diagnosis probe apparatus comprising:
a transmission signal forming unit forming a transmission signal for obtaining a frame of an ultrasound image;
an ultrasound probe transducing the transmission signal of the transmission signal forming unit into an ultrasound signal, transmitting the ultrasound signal to an object, and obtaining analog ultrasound data reflected from the object;
a two-dimensional array processor adjacently arranging the obtained analog ultrasound data compensated with respect to time gains thereof and adjusted with respect to intensity and contrast thereof for each ultrasound vector to be processed into two-dimensional array ultrasound data;
a compressor compressing the two-dimensional array ultrasound data adjacently arranged for each ultrasound vector; and
a wireless communication unit wirelessly transmitting the compressed two-dimensional array ultrasound data to an ultrasound diagnostic apparatus.

2. The apparatus of claim 1, wherein the two-dimensional array processor arranges received ultrasound vectors in a serial stream to be adjacent for each ultrasound vector unit lengthwise to be processed into the two-dimensional array ultrasound data.

3. The apparatus of claim 1, further comprising:
a beam former generating digitalized ultrasound data by using the analog ultrasound data obtained from the ultrasound probe;
a time gain compensation (TGC) unit compensating the digitalized ultrasound data with time gains; and
an intensity and contrast adjuster adjusting intensity and contrast of the ultrasound image.

4. The apparatus of claim 1, further comprising:
a TGC unit compensating the analog ultrasound data obtained from the ultrasound probe with time gains;
a beam former generating digitalized ultrasound data by using the ultrasound data compensated with time gains; and
an intensity and contrast adjuster adjusting intensity and contrast of the ultrasound image.

5. The apparatus according to claim 3, wherein the beam former generates ultrasound data comprising M number of arrays whose size is N when using M number of ultrasounds for one ultrasound image frame and performing N times of sampling each ultrasound that is reflected from the object and returns.

6. The apparatus according to claim 3, wherein the TGC unit compensates the ultrasound data according to a TGC table.

7. The apparatus according to claim 3, wherein the intensity and contrast adjuster adjusts an intensity value of a certain value or less to 0 and adjusts an intensity value of a certain value or more to a maximum value.

8. The apparatus according to claim 3, wherein the intensity and contrast adjuster adjusts a contrast value of a certain value or less to 0 and adjusts a contrast value of a certain value or more to a maximum value.

9. The apparatus of claim 1, wherein the two-dimensional array processor generates two-dimensional ultrasound data comprising N×M arrays when using M number of ultrasounds for one ultrasound image frame and performing N times of sampling each ultrasound that is reflected from the object and returns.

10. The apparatus of claim 1, wherein the wireless communication unit comprises short-distance wireless communication by using one of Bluetooth, a wireless universal serial bus (USB), a wireless local area network (LAN), wireless fidelity (WiFi), Zigbee, and infrared data association (IrDA).

11. A mobile ultrasound diagnostic system comprising:
a mobile ultrasound diagnosis probe apparatus, which is portable, digital-processes ultrasound data obtained from an object, compensates the digitalized ultrasound data with time gains, adjusts intensity and contrast of the ultrasound data, processes the ultrasound data into two-dimensional array ultrasound data by adjacently arranging for each ultrasound vector, compresses the two-dimensional array ultrasound data, and wirelessly transmits the two-dimensional array ultrasound data, and
an ultrasound diagnostic apparatus, which receives the two-dimensional array ultrasound data from the mobile ultrasound diagnosis probe apparatus and generates ultrasound image data for diagnosis by releasing compression of the two-dimensional array ultrasound data.

12. The system of claim 11, wherein the mobile ultrasound diagnosis probe apparatus arranges received ultrasound vectors in a serial stream to be adjacent for each ultrasound vector unit lengthwise to be processed into the two-dimensional array ultrasound data.

13. The system of claim 11, wherein the ultrasound diagnostic apparatus determines an ultrasound measurement depth according to an input of a user and transmits a parameter for adjusting time gains and a parameter for adjusting the intensity and the contrast to the mobile ultrasound diagnosis probe apparatus.

14. The system of claim 11, wherein the ultrasound diagnostic apparatus transmits dummy data for automatically measuring a wireless communication environment and for determining the size of transmission data to the mobile ultrasound diagnosis probe apparatus, and
wherein the mobile ultrasound diagnosis probe apparatus receives the dummy data from the ultrasound diagnostic apparatus, calculates an available band of wireless communication currently used by measuring an amount of time used to receive the dummy data, and determines the size of data to be wirelessly transmitted, according to the available band.

15. The apparatus according to claim 4, wherein the beam former generates ultrasound data comprising M number of arrays whose size is N when using M number of ultrasounds for one ultrasound image frame and performing N times of sampling each ultrasound that is reflected from the object and returns.

16. The apparatus according to claim 4, wherein the TGC unit compensates the ultrasound data according to a TGC table.

17. The apparatus according to claim 4, wherein the intensity and contrast adjuster adjusts an intensity value of a certain value or less to 0 and adjusts an intensity value of a certain value or more to a maximum value.

18. The apparatus according to claim 4, wherein the intensity and contrast adjuster adjusts a contrast value of a certain value or less to 0 and adjusts a contrast value of a certain value or more to a maximum value.
